(19) United States
(12) Patent Application Publication
(10) Pub. No.: US 2006/0081469 A1
(43) Pub. Date: Apr. 20, 2006

(54) Battery pack of a mobile communication terminal to be capable of reading output of bio-sensors and self-diagnosis system

(75) Inventor: Min-Hwa Lee, Seoul (KR)

Correspondence Address: Jenkens & Gilchrist, a Professional;Corporation, Suite 900, 901 15th Street, Washington, DC, 20005, US

(73) Assignee: Healthpia America Co., Ltd., Newark, NJ (US)

(21) Appl. No.: 11/284,953

(22) Filed: Nov. 23, 2005

Related U.S. Application Data
(63) Continuation of application No. PCT/KR03/01927, filed on Sep. 19, 2003.

(30) Foreign Application Priority Data
May 29, 2003 (KR) 10-2003-0034524

(51) Int. Cl. G01N 33/487; C12Q 1/00

(52) U.S. Cl. 204/403.02; 204/403.04; 429/61

(57) ABSTRACT

A battery pack for self-diagnosis and a system using the same, which can read a data value from a body fluid sensor (referred to as a biosensor) reacting with body fluid such as urine, and indicate a measurement value of a test item such as a blood glucose level, a cholesterol level or etc. The battery pack includes: a power supply for supplying electric power to a working electrode of the body fluid sensor; a current detector for detecting the amount of electric current flowing into the working electrode; a battery pack controller for controlling an electric power supply operation for the working electrode, reading, from a memory, the test item-based measurement value corresponding to the detected current amount, and outputting the read measurement value; and an interface for carrying out an interface function so that the test item-based measurement value outputted from the battery pack controller can be sent to the main body of the mobile communication terminal.

The present invention claims the benefit of international patent application number PCT/KR2003/001927 in Korea on Sep. 19, 2003 and Korean patent application no. 10-2003-0034524, which are both hereby incorporated by reference in their entirety.

### TECHNICAL FIELD

The present invention relates to a battery pack provided in a mobile communication terminal, and more particularly to a battery pack for self-diagnosis and a self-diagnosis system using the same, which can measure and indicate a blood glucose level, a cholesterol level, etc.

### BACKGROUND ART

As interest in health increases, self-diagnosis kits capable of performing self-diagnosis in a general home setting or portable self-diagnosis kits are variously developed. There are biosensors for measuring a blood glucose level as representative self-diagnosis kits. As the biosensors are disclosed in many patent publications, each of the biosensors includes an electrode unit including a plurality of electrodes formed by screen printing on an electrical insulating substrate, etc. and an enzyme reaction layer, prepared on the electrode unit, including a water-soluble polymer, an oxidation-reduction enzyme and an electron acceptor.

Operation of a biosensor for measuring a blood glucose level will now be described. First, if a blood sample is introduced into an enzyme reaction layer, blood glucose is oxidized by glucose oxidase, and the glucose oxidase is reduced. The glucose oxidase oxidizes an electron acceptor, and the electron acceptor is reduced. The reduced electron acceptor is electrochemically re-oxidized while its electrons are lost from an electrode surface by predetermined voltage. Since glucose concentration of the blood sample is proportionate to an amount of electric current while the electron acceptor is oxidized, the biosensor can measure the blood glucose concentration by measuring the amount of electric current.

A self-diagnosis kit includes a biosensor as consumption goods reacting with sample fluid, and a biosensor reader for reading a result of the reaction from the biosensor and externally indicating the read reaction result. There are problems in that the economic burden or cost increases since the biosensor reader must be additionally provided when the self-diagnosis kit is used and it is difficult for the self-diagnosis kit to be carried.

If the biosensor can be embedded in a mobile communication terminal as portable equipment, the inconvenience of separately carrying the biosensor reader can be addressed.

It is seriously needed that a system capable of minimizing the economic burden or cost is developed since the economic burden imposed on users can be high where a mobile communication terminal equipped with the biosensor reader embedded therein is newly purchased.

### DISCLOSURE OF THE INVENTION

Therefore, the present invention has been made in view of the above problems, and it is one object of the present invention to provide a battery pack for self-diagnosis provided in a mobile communication terminal, which can read a value from a body fluid sensor reacting with a specified component contained in blood, urine, etc., and output and indicate the read value as self-diagnosis data.

It is another object of the present invention to provide a self-diagnosis system, which can indicate and remotely send self-diagnosis data read from a body fluid sensor without changing the design of a main body of a mobile communication terminal.

### BRIEF DESCRIPTION OF THE DRAWINGS

The above and other objects, features and other advantages of the present invention will be more clearly understood from the following detailed description taken in conjunction with the accompanying drawings, in which:

FIG. 1 is a block diagram illustrating a battery pack and its peripheral units in accordance with an embodiment of the present invention;

FIG. 2 is a perspective view illustrating a slot into which a body fluid sensor is inserted formed in an external surface of a battery pack case in accordance with an embodiment of the present invention;

FIGS. 3A and 3B are plane and rear views illustrating the body fluid sensor capable of being coupled to the battery pack in accordance with an embodiment of the present invention;

FIG. 4 is a sectional view illustrating the body fluid sensor shown in FIG. 1; and

FIG. 5 is a circuit diagram illustrating a current detector shown in FIG. 1.

### BEST MODE FOR CARRYING OUT THE INVENTION

In accordance with an embodiment of the present invention, the above and other objects can be accomplished by the provision of a battery pack for self-diagnosis including a slot coupled to a body fluid sensor equipped with an electrode unit having a working electrode and a reference electrode and an enzyme reaction layer formed on the electrode unit, comprising:

a power supply for supplying electric power to the working electrode of the body fluid sensor coupled to the slot;

a current detector for detecting an amount of electric current flowing into the working electrode;

a battery pack controller for controlling an electric power supply operation for the working electrode, reading, from a memory, a test item-based measurement value corresponding to the detected current amount, and outputting the read measurement value; and

an interface for carrying out an interface function so that the test item-based measurement value outputted from the battery pack controller can be sent to a mobile communication terminal.

Thus, the present invention can perform a self-diagnosis operation for a cholesterol level, a blood glucose level, etc. using body fluid by means of a mobile communication terminal without an additional reader.

In accordance with another embodiment of the present invention, there is provided a self-diagnosis system, comprising:

a battery pack coupled to a body fluid sensor equipped with an electrode unit having a working electrode and a reference electrode and an enzyme reaction layer formed on the electrode unit, wherein the battery pack includes an external surface in which a slot is formed so that the body fluid sensor can be inserted, supplies electric power to the working electrode, and outputs a test item-based measurement value corresponding to an amount of electric current flowing into the working electrode of the body fluid sensor; and

a main body of a mobile communication terminal including a controller for commanding the battery pack to measure a test item according to a user's request, reading, from a memory, the test item-based measurement value corresponding to the amount of electric current sent from the battery pack, and outputting the read measurement value.

In view of configuration, the self-diagnosis system can be designed so that the battery pack detects the amount of electric current flowing into the working electrode of the body fluid sensor to send the detected current amount to the terminal's main body and the terminal's main body indicates the test item-based measurement value corresponding to the detected current amount.

Now, preferred embodiments of the present invention will be described in detail with reference to the annexed drawings so that those skilled in the art can easily understand the present invention.

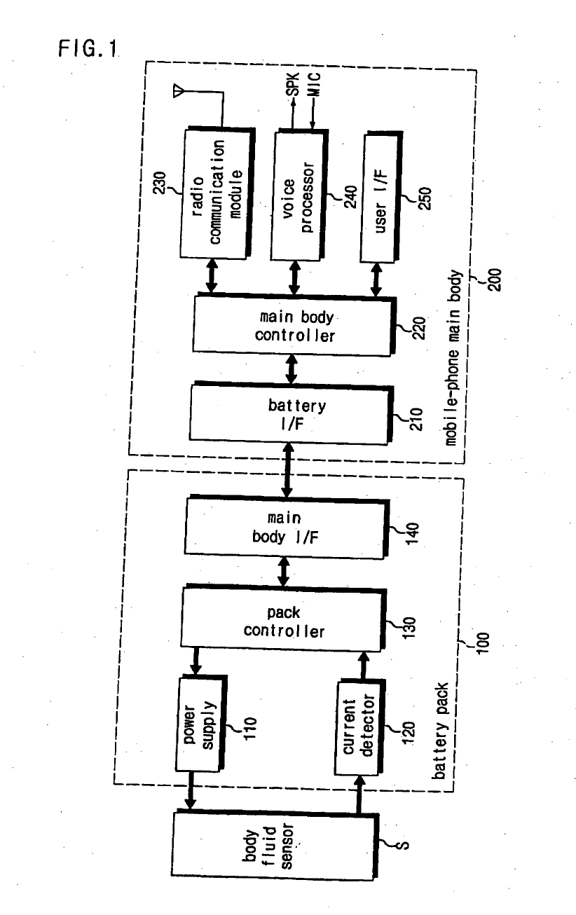

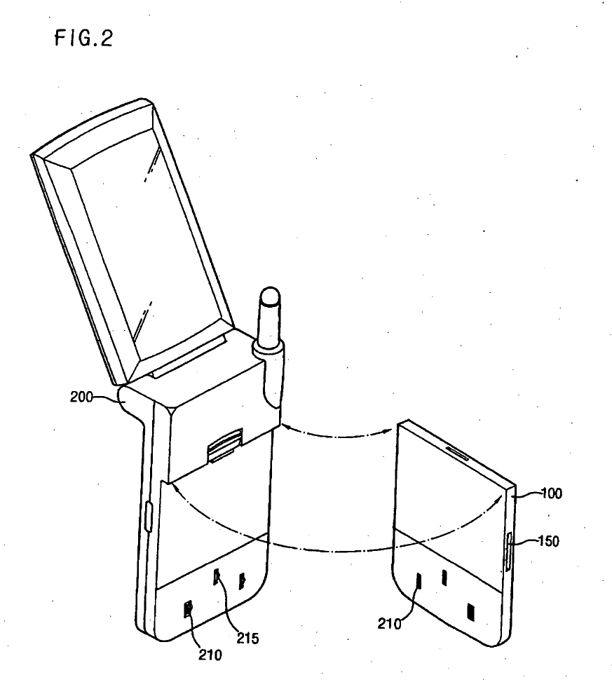

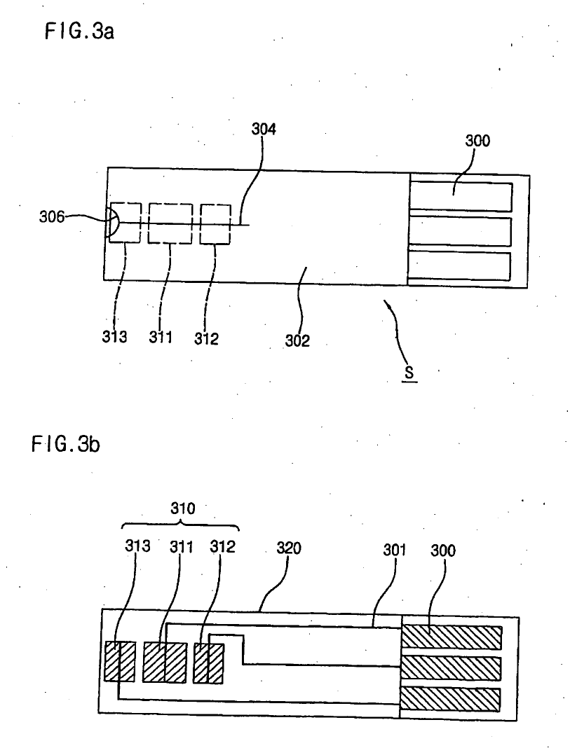

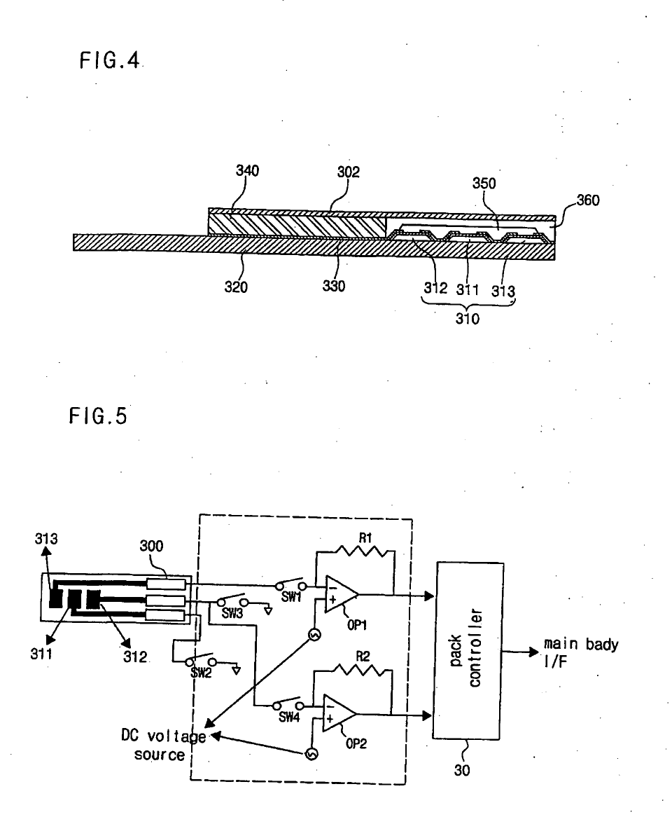

FIG. 1 is a block diagram illustrating a state in which a battery pack 100 and a main body 200 of a mobile communication terminal are coupled to each other in accordance with an embodiment of the present invention. FIG. 2 is a perspective view illustrating a slot 150 into which a body fluid sensor S is inserted formed in an external surface of the battery pack 100 in accordance with an embodiment of the present invention. FIGS. 3A and 3B are plane and rear views illustrating the body fluid sensor S capable of being coupled to the battery pack 100 in accordance with an embodiment of the present invention. FIG. 4 is a sectional view illustrating the body fluid sensor S shown in FIG. 1. FIG. 5 is a circuit diagram illustrating a current detector 110 shown in FIG. 1.

First, referring to FIG. 1, the battery pack 100 for self-diagnosis includes a power supply 110, a current detector 120, a pack controller 130 and a main body interface (I/F) 140.

The power supply 110 performs an operation for supplying electric power to working electrodes of the body fluid sensor S coupled to the slot 150 for the body fluid sensor S. The current detector 120 detects amounts of electric currents flowing into the working electrodes of the body fluid sensor S.

The power supply 110 includes a plurality of battery cells serving as direct current (DC) voltage sources. For reference, it can be assumed that the slot 150 for the body fluid sensor S is formed in one side surface of an external case for the battery pack 100 as shown in FIG. 2. The body fluid sensor S is referred to as a biosensor. The slot 150 for the body fluid sensor S will be described below.

As shown in FIG. 3A, leader terminals 300 are formed at one end of an electrical insulating substrate for the body fluid sensor S. The number of leader terminals 300 is the same as the number of electrodes. The leader terminals 300 are connected to electrodes 311, 312 and 313 formed at the other end of the body fluid sensor S through leader lines 301 as shown in FIG. 3B. Furthermore, a slit 304 is formed in a cover 302 for the body fluid sensor S as shown in FIG. 3A. The slit 304 is extended from a concave groove 306 to the electrodes 311, 312 and 313 formed at one end of the cover 302. The slit 304 serves as an air outlet where a vital sample (e.g., blood, urine, saliva, etc.) is injected thereto according to a capillary phenomenon.

For reference, the electrical insulating substrate for the body fluid sensor S can be formed by a polymer substrate typically manufactured using nonconductive materials such as a polyethylene terephthalate resin, a polyvinyl chloride resin and a polycarbonate resin. A leader unit including the leader lines 301 and the leader terminals 300 can be formed by a typical screen-printing method. In the electrodes 311, 312 and 313, the reference numeral 311 denotes a reference electrode, and the reference numerals 312 and 313 denote working electrodes. The electrodes 311, 312 and 313 are used for detecting amounts of electric currents generated at the time of electron acceptor oxidation or reduction on an enzyme reaction layer. The reference electrode 311 is located between the working electrodes 312 and 313. This arrangement is set to detect the current amounts between the reference electrode 311 and the working electrodes 312 and 313 adjacent thereto. In accordance with the embodiment of the present invention, the battery pack 100 can detect the current amounts between the reference electrode 311 and the working electrodes 312 and 313 and indicate measurement values of a test item.

In order for the electrodes 311, 312 and 313 to be insulated therebetween, an insulating layer 330 is formed as an insulating material is printed or coated on the remaining areas other than predetermined areas of upper parts of the electrode 311, 312 and 313 as shown in FIG. 4. As the insulating material, a nonconductive screen printing ink or insulating ink can be used. An enzyme reaction layer 350 is formed on exposed upper parts of the electrodes 311, 312 and 313 after the insulating material is printed or coated and an upper part of the insulating layer 330. The enzyme reaction layer 350 includes enzymes reacting with an injected body fluid and an electron acceptor. For example, the enzyme reaction layer 350 must contain different enzymes according to types of test items such as a cholesterol level, a blood glucose level, etc. One example is shown in the following Table 1.

If the body fluid sensor S is a sensor for measuring a blood glucose level, the enzyme reaction layer 350 contains glucose oxidase as shown in the following Table 1. If a blood sample being body fluid is introduced into the enzyme reaction layer 350, blood glucose is oxidized by the glucose oxidase, and the glucose oxidase is reduced. Here, the glucose oxidase oxidizes an electron acceptor and then the electron acceptor is reduced. The reduced electron acceptor is electrochemically re-oxidized as its electrons are lost from an electrode surface by predetermined voltage. For reference, as glucose concentration within the blood sample is proportionate to an amount of electric current generated while the electron acceptor is oxidized, the amount of electric current is measured through the leader terminals 301 and the measurement value of a test item corresponding to the measured amount of electric current, i.e., a value of the glucose concentration, can be produced.

TABLE 1

| Component to be | |
|---|---|
| analyzed | Enzyme |
| Glucose | Glucose oxidase |
| Cholesterol | Cholesterol esterase |
| | Cholesterol oxidase |
| | Peroxinase |
| Creatinine | Creatininase |
| | Creatinase |
| | Sarcosine oxidase |
| Lactic acid | Lactate oxidase |

Finally, the cover 302 is adhered to an upper surface of a spacer 340 for the body fluid sensor S as shown in FIG. 4, and the slit 304 is formed so that air can be eliminated from a body-fluid injection space 360 formed by the adhesion between the spacer 340 and the cover 302. As shown in FIG. 3A, the slit 304 is extended by predetermined length in a direction of the electrodes 311, 312 and 313. Furthermore, the slit 304 must be extended up to the upper parts of the electrodes 311, 312 and 313. The reason is to allow the vital sample to be stably introduced up to the electrode 312.

The biosensor having two working electrodes and one reference electrode has been described as an example of the body fluid sensor S, but the biosensor having one working electrode and one reference electrode can be used as a body fluid sensor.

The configuration of a current detector 120 for detecting amounts of electric currents flowing into the working electrodes of the body fluid sensor S will be described in detail with reference to FIG. 5.

First, the current detector 120 includes operational amplifiers OP1 and OP2 used for current-voltage converters and switches SW1˜SW4. Non-inversion input terminals (+) of the operational amplifiers OP1 and OP2 are connected to a DC voltage source being a power supply 110, respectively, and inversion input terminals (−) of the operational amplifiers OP1 and OP2 are coupled to one side of the first switch SW1 and one side of the fourth switch SW4, respectively. Other sides of the switches SW1 and SW4 can be coupled to leader terminals 300 connected to the first working electrode 313 and the second working electrode 312 for the body fluid sensor's stripe. The operational amplifiers OP1 and OP2 supply electric power to the working electrodes 313 and 312, detect amounts of electric currents flowing into the working electrodes 313 and 312 according to electric power supply, and output voltage values.

The reference electrode 311 of the body fluid sensor S is grounded through the second switch SW2, and a leader terminal connected to the second working electrode 312 of the body fluid sensor S is grounded through the third switch SW3. The switches SW1˜SW4 are turned on/off by the control of a pack controller 130 and used for controlling electric current paths for a circuit.

Referring to FIG. 1, the pack controller 130 controls electric power supply for the working electrodes 312 and 313 according to the control of a mobile-phone main body 200. The pack controller 130 reads, from an internal memory, test item-based measurement values corresponding to the amounts of electric currents detected from the working electrodes 312 and 313, and produces and outputs an average value of the measurement values. If a single working electrode is provided, the pack controller 130 controls electric power supply to be supplied to the single working electrode, detects an amount of electric current flowing into the working electrode, reads a test item-based measurement value from an internal memory, and outputs the read measurement value. For reference, the internal memory of the pack controller 130 stores a table based on test items such as a cholesterol level and a blood glucose level. The table has a form in which test item-based measurement values are mapped to detected amounts of electric currents. The detected amounts of electric currents can be expressed as voltage values inputted into an input terminal of the pack controller 130 after an analog/digital (A/D) conversion.

A main body interface (I/F) 140 performs an interface function between the battery pack controller 130 and the mobile-phone main body 200 so that a test item-based measurement value outputted from the battery pack controller 130 can be sent to the mobile-phone main body 200. As shown in FIG. 2, the main body I/F 140 can perform a communication operation by connecting a special communication terminal 215 to the mobile-phone main body 200 and the battery pack 100. The interface function can be replaced with a power-line communication operation using an existing power output terminal 210.

The configuration in which the battery pack 100 is coupled to the mobile-phone main body 200 will be described. First, the mobile-phone main body 200 commands the battery pack 100 to perform a test item-based measurement operation according to a user's request. In response to the user's request, the mobile-phone main body 200 indicates a test item-based measurement value sent from the battery pack 100. Alternatively, the mobile-phone main body 200 commands the battery pack 100 to perform the test item-based measurement operation according to the user's request. In response to the user's request, the mobile-phone main body 200 can read, from an internal memory, a test item-based measurement value corresponding to an amount of electric current sent from the battery pack 100 and output the read measurement value.

The configuration of the mobile-phone main body 200 will now be described. A radio communication module 230 modulates a test item-based measurement value or communication data outputted from a main body controller 220 and performs a frequency conversion operation. Then, the radio communication module 230 transmits a radio signal through an antenna ANT. The radio communication module 230 separates a radio signal received through the antenna ANT into voice data and another signal. The radio communication module 230 performs a frequency conversion and demodulation operation for the separated radio signal and sends a result of the frequency conversion and demodulation operation to the main body controller 220.

A voice processor 240 digitally processes voice data inputted from a microphone MIC under the control of the main body controller 220 and sends the digitally processed voice data to the radio communication module 230. The voice processor 240 demodulates the voice data received through the radio communication module 230 and outputs the demodulated voice data through a speaker SPK.

On the other hand, the main body controller 220 outputs guidance information for test item-based measurement through a user interface (I/F) 250, sends a test item selected by the user and a test item-based measurement command to the battery pack 100. The main body controller 220 performs a control operation so that the test item-based measurement value sent from the battery pack 100 can be outputted through the user I/F 250 or the radio communication module 230. Alternatively, the main body controller 220 can receive data indicating an amount of electric current flowing into the single working electrode from the battery pack 100, read, from the internal memory, a test item-based measurement value corresponding to the amount of the electric current, and output the read measurement value. Alternatively, the main body controller 220 can receive data indicating amounts of electric currents flowing into the two working electrodes, read test item-based measurement values corresponding to the amounts of the electric currents, produces an average value of the measurement values, and output the produced average value.

The battery pack I/F 210 coupled to the main body I/F 140 interfaces data transmitted and received between the battery pack 100 and the mobile-phone main body 200.

Operation of the battery pack 100 capable of performing a self-diagnosis operation for various test items through the body fluid sensor S reacting with the body fluid will now be described.

First, the user desiring to perform the self-diagnosis operation allows the body fluid such as blood to come into contact with the concave groove 306 of the body fluid sensor S, and inserts the body fluid sensor S into the slot 150. At this time, the pack controller 130 determines whether the body fluid sensor S has been inserted into the slot 150. A test operation can be performed when a switch located within the slot 150 for the body fluid sensor S is short-circuited and hence input voltage is dropped to 0V.

When the body fluid sensor S is inserted, an operating mode of the pack controller 130 is switched to a detection mode as a result of the body fluid reaction, and electric power is supplied to the first working electrode 313. In this case, an electric power supply operation can be implemented as the first and second switches SW1 and SW2 are turned on and the third and fourth switches SW3 and SW4 are turned off.

If the body fluid has arrived at the first working electrode 313 and the reference electrode 311, electric current flows between the two electrodes 311 and 313 through the reaction of the enzyme reaction layer 350. The electric current flowing into the first working electrode 313 is converted into electric voltage by a resister R1 connected to the output terminal and the inversion input terminal (−) provided in the operational amplifier OP1. The electric voltage is inputted into the pack controller 130, and is converted into digital data. Accordingly, the pack controller 130 can detect the voltage value converted into the digital data, that is, an amount of electric current flowing into the first working electrode 313. When the electric current flowing into the first working electrode 313 is detected, the pack controller 130 starts a timing operation. The timing operation is performed to measure the time taken to detect the electric current flowing into the second working electrode 312. A measured time value is used for determining whether body fluid has been appropriately injected.

After detecting the amount of electric current flowing into the first working electrode 313 and starting the timing operation, the pack controller 130 supplies electric power to the second working electrode 312. An operation for supplying the electric power to the second working electrode 312 can be implemented when the second and fourth switches SW2 and SW4 are turned on and the first and third switches SW1 and SW3 are turned off. The reason why the electric power is supplied to the second working electrode 312 is to determine whether the body fluid, i.e., the sample, has appropriately arrived at the second working electrode 312. When the second and fourth switches SW2 and SW4 are in an OFF state and the first and third switches SW1 and SW3 are in an ON state, the determination can be made as to whether the body fluid has appropriately arrived at the second working electrode 312. In this case, the second working electrode 312 serves as a reference electrode.

As described above, after the electric power is supplied to the second working electrode 312, the pack controller 130 detects an amount of electric current flowing into the second working electrode 312. Furthermore, if the amount of electric current flowing into the second working electrode 312 is detected, a determination is made as to whether a measured time value (associated with reaction times of the working electrodes 313 and 312) between a time-point of detecting the amount of electric current flowing into the first working electrode 313 and a time-point of detecting the amount of electric current flowing into the second working electrode 313 is within a predetermined threshold range. According to a result of the determination, the pack controller 130 can determine whether the body fluid injection is erroneous. Furthermore, the pack controller 130 checks amounts of electric currents flowing into the working electrodes 313 and 312 and can determine an error of manufactured electrodes. In other words, if an area of any one electrode is widely formed due to a manufacturing error, an electric current difference between the erroneous electrode and another electrode can increase.

Thus, the pack controller 130 can detect an error of the manufactured body fluid sensor by comparing the amounts of electric currents flowing into the working electrodes 313 and 312.

Upon determining that an erroneous sample injection has occurred or any one electrode error is present by checking the reaction times of the working electrodes 313 and 312 and the detected amounts of electric currents, the pack controller 130 outputs a signal indicating an error state through the mobile-phone main body 200. On the other hand, if the body fluid injection is appropriate and the working electrodes are appropriately manufactured, the pack controller 130 turns off the first, second, third and fourth switches during a predetermined incubation time. The reason why the incubation time is used is to obtain uniform electrode reactions. This incubation time is not necessarily required. Furthermore, when the user desires to obtain a quick measurement result, the first, second and fourth switches are maintained in an ON state and the third switch is maintained in an OFF state.

After a predetermined time (or incubation time), the pack controller 130 sequentially supplies the electric power to the first working electrode 313 and the second working electrode 312. Then, the pack controller 130 detects an amount of electric current flowing between the first working electrode 313 and the reference electrode 311 and an amount of electric current flowing between the second working electrode 312 and the reference electrode 311 after a predetermined time. As described above, a switch control operation must be performed to supply the electric power to the working electrodes and to detect their current amounts.

After detecting the amount of electric current flowing into the first working electrode 313 and the amount of electric current flowing into the second working electrode 312 in a state where the first, second and fourth switches are turned on, the pack controller 130 reads, from the internal memory, test item-based measurement values corresponding to the detected current amounts. Then, the pack controller 130 produces an average value of the read measurement values and sends the average value to the mobile-phone main body 200. Thus, the controller 220 of the mobile-phone main body 200 enables the user I/F 250 (or an indicator, a display unit, etc.) to indicate the average value or sends the average value to a remote server or terminal by radio.

Thus, the user can perform a self-diagnosis operation through a mobile communication terminal to confirm a cholesterol level, a blood glucose level, etc. Of course, self-diagnosis data can be remotely transmitted, and hence a remote diagnosis service can be provided.

The self-diagnosis operation for measuring a blood glucose level through blood has been described in the embodiments of the present invention, but a self-diagnosis operation based on test items through blood, e.g., a glutamate oxaloacetate transaminase (GOT) test, a lactate dehydroganase (LDH) test, a leusine amino peptidase test, an alkaline phosphatase (ALP) test, a thymol turbidity test (TTT), a zinc turbidity test (ZTT), a urea nitrogen test, a neutral fat test, a hemoglobin test, etc. can be performed. A urine protein test, a urine sugar test, an occult blood reaction test, a urobilinogen test, a urinary bilirubin test, a human chorionic gonadotropin (HCG) test, etc. can be performed through the self-diagnosis operation using urine.

### INDUSTRIAL APPLICABILITY

As apparent from the above description, the present invention can read a reaction value from a biosensor through a battery pack provided in a mobile communication terminal, perform a self-diagnosis operation for measuring a cholesterol level, a blood glucose level, etc. using the battery pack without an additional reader, and conveniently perform various tests associated with diseases through urine by means of the mobile communication terminal or the battery pack coupled to the mobile communication terminal.

Although the preferred embodiments of the present invention have been disclosed for illustrative purposes, those skilled in the art will appreciate that various modifications, additions and substitutions are possible, without departing from the scope of the invention.

For example, the battery pack 100 for reading data from a body fluid sensor equipped with two working electrodes has been described in the embodiment of the present invention, but the battery pack 100 can be designed to read data from a body fluid sensor equipped with one working electrode. In this case, the pack controller 130 can simply apply voltage to the working electrode, detect an amount of electric current flowing into the working electrode, and send a test item-based measurement value corresponding to the detected current amount to the mobile-phone main body 200.

Furthermore, there has been described a method in which the battery pack 100 reads the test item-based measurement value corresponding to the detected current amount and sends the read measurement value to the mobile-phone main body 200 in accordance with the embodiment of the present invention, but the self-diagnosis system can be designed so that the battery pack 100 only detects the amount of electric current flowing into the working electrode, and the mobile-phone main body 200 can read the test item-based measurement value corresponding to the detected current amount and output the read measurement value. In these cases, the self-diagnosis system can be designed so that only one test item can be measured. Furthermore, the self-diagnosis system can be designed so that a plurality of test items associated with a cholesterol level test, a blood glucose level test, a liver function test, etc. can be selectively measured. A user interface must be provided so that a user can select any one of the test items to measure the plurality of test items, and a table necessary for measuring the selectable test items must be stored in a memory.

Furthermore, the operation of the pack controller 130 for determining whether the body fluid sensor S has been appropriately inserted and for applying electric power to a working electrode has been described in accordance with the embodiment of the present invention, but the self-diagnosis system can be designed so that the electric power is applied to the working electrode when a test item-based measurement command is inputted from the mobile-phone main body 200.

The present invention is not limited to the above-described embodiments, but the present invention is defined by the claims which follow, along with their full scope of equivalents.

1. A battery pack for self-diagnosis capable of being coupled to a body fluid sensor equipped with an electrode unit having a working electrode and a reference electrode and an enzyme reaction layer formed on the electrode unit, comprising:
a power supply for supplying electric power to the working electrode of the body fluid sensor;
a current detector for detecting an amount of electric current flowing into the working electrode;
a battery pack controller for controlling an electric power supply operation for the working electrode, reading, from a memory, a test item-based measurement value corresponding to the detected current amount, and outputting the read measurement value; and
an interface for carrying out an interface function so that the test item-based measurement value outputted from the battery pack controller can be sent to a mobile communication terminal.

2. A battery pack for self-diagnosis capable of being coupled to a body fluid sensor equipped with an electrode unit having working electrodes and a reference electrode and an enzyme reaction layer formed on the electrode unit, comprising:
a power supply for supplying electric power to the working electrodes of the body fluid sensor;
a current detector for detecting amounts of electric currents flowing into the working electrodes;
a battery pack controller for controlling electric power supply operations for the working electrodes, reading, from a memory, test item-based measurement values corresponding to the detected current amounts, and producing an average value of the read measurement values; and
an interface for carrying out an interface function so that the produced average measurement value from the battery pack controller can be sent to a mobile communication terminal.

3. A self-diagnosis system, comprising:
a battery pack coupled to a body fluid sensor equipped with an electrode unit having a working electrode and a reference electrode and an enzyme reaction layer formed on the electrode unit, wherein the battery pack includes an external surface in which a slot is formed so that the body fluid sensor can be inserted, supplies electric power to the working electrode, and outputs a test item-based measurement value corresponding to an amount of electric current flowing into the working electrode of the body fluid sensor; and
a main body of a mobile communication terminal including a controller for commanding the battery pack to measure a test item according to a user's request and indicating the test item-based measurement value sent from the battery pack in response to the user's request.

4. The self-diagnosis system as set forth in claim 3, wherein the battery pack comprises:
a power supply for supplying electric power to the working electrode of the body fluid sensor;
a current detector for detecting the amount of electric current flowing into the working electrode;
a battery pack controller for controlling an electric power supply operation for the working electrode, reading, from a memory, the test item-based measurement value corresponding to the detected current amount, and outputting the read measurement value; and
an interface for carrying out an interface function so that the test item-based measurement value outputted from the battery pack controller can be sent to the main body of the mobile communication terminal.

5. The self-diagnosis system as set forth in claim 3, wherein the controller performs a control operation so that the test item-based measurement value can be sent to any one of a specified terminal and a remote server according to the user's request.

6. A self-diagnosis system, comprising:
a battery pack coupled to a body fluid sensor equipped with an electrode unit having a working electrode and a reference electrode and an enzyme reaction layer formed on the electrode unit, wherein the battery pack includes an external surface in which a slot is formed so that the body fluid sensor can be inserted, supplies electric power to the working electrode, and outputs a test item-based measurement value corresponding to an amount of electric current flowing into the working electrode of the body fluid sensor; and
a main body of a mobile communication terminal including a controller for commanding the battery pack to measure a test item according to a user's request, reading, from a memory, the test item-based measurement value corresponding to the amount of electric current sent from the battery pack, and outputting the read measurement value.
